(19) United States
(12) Patent Application Publication
(10) Pub. No.: US 2016/0354297 A1
(43) Pub. Date: Dec. 8, 2016

(54) COSMETIC COMPOSITION COMPRISING AT LEAST ONE SPECIFIC SILANE, AT LEAST ONE ANIONIC AND/OR NON-IONIC POLYSACCHARIDE AND AT LEAST ONE WATER-SOLUBLE MINERAL SALT

(71) Applicant: L'OREAL, Paris (FR)

(72) Inventors: Marie-Cécile DEGOUL, Paris (FR); Zhenhua SONG, Paris (FR)

(21) Appl. No.: 15/038,837

(22) PCT Filed: Nov. 24, 2014
(86) PCT No.: PCT/EP2014/075418

§ 371 Date: May 24, 2016

(30) Foreign Application Priority Data
Nov. 25, 2013 (FR) 1361584

(51) Int. Cl. A61K 8/58; A61Q 5/06; A61K 8/86; A61Q 5/12; A61K 8/73; A61K 8/19

(52) U.S. Cl. CPC A61K 8/585; A61K 8/73; A61K 8/19; A61K 8/86; A61K 8/731; A61K 8/732; A61Q 5/12; A61Q 5/06; A61K 2800/52

(57) ABSTRACT

The invention relates to a cosmetic composition comprising: (a) one or more specific silanes, (b) one or more polysaccharides chosen from anionic or non-ionic polysaccharides, and their mixtures, (c) one or more water-soluble mineral salts. The invention also relates to a method for the cosmetic treatment of keratinous fibres, in particular human keratinous fibres, such as the hair, in which the cosmetic composition as defined above is applied to the said fibres. Finally, the invention relates to the use of the above composition in the treatment of keratinous fibres and in particular in the conditioning and/or care of keratinous fibres, preferably the hair.

The present invention relates to a cosmetic composition comprising one or more specific silanes, one or more anionic and/or non-ionic polysaccharides and/one or more water-soluble mineral salts intended for the treatment of keratinous fibres, in particular human keratinous fibres, such as the hair.

The present invention also relates to a method for the cosmetic treatment of keratinous fibres employing the said composition.

Finally, the invention relates to the use of this composition in the cosmetic treatment of keratinous fibres and in particular the hair.

The cosmetic compositions for the treatment of keratinous fibres are generally applied to sensitized hair, that is to say hair which is generally damaged or embrittled by the action of external atmospheric agents, such as light and bad weather, and/or mechanical or chemical treatments, such as brushing, combing, dyeing operations, bleaching operations, permanent waves and/or straightening operations.

It is known, in order to improve the cosmetic properties of these compositions, to introduce therein cosmetic agents, known as conditioning agents, intended mainly to repair or to limit the harmful or undesirable effects brought about by the various treatments or attacks to which hair fibres are more or less repeatedly subjected. These conditioning agents may, of course, also improve the cosmetic behaviour of natural hair.

With this aim, it has already been proposed to use cosmetically active organic compounds, such as cationic polymers and silicones, as conditioning agents in care cosmetic compositions, such as conditioners, in order to confer satisfactory cosmetic properties on the hair, in particular sheen, softness, suppleness, lightness, a natural feel and an improved ability to be disentangled.

However, the use of these compounds in care and/or conditioning cosmetic compositions does not provide the hair with satisfactory and lasting styling properties. This is because these compositions generally provide styling effects, such as effects of form retention, of body and/or of manageability of the hair, which remain insufficient and which have a tendency to fade away after washing the hair with a standard shampoo.

In point of fact, consumers are increasingly in search of care compositions which are not only capable of appropriately conditioning the hair but also capable of providing satisfactory and lasting styling effects.

In order to meet these requirements, compositions intended for caring for and/or conditioning the hair have been developed which comprise organosilicon compounds, such as alkoxysilanes, for example. These care compositions make it possible to condition the hair, in particular by providing it with a satisfactory soft, clean and natural feel, while conferring pronounced and lasting styling effects.

In particular, these compositions have proved to be particularly advantageous as they make it possible to facilitate the shaping of fine hair and to confer advantageous styling effects on curly or very curly hair, especially by improving the design and the control of the curls.

However, compositions comprising alkoxysilanes often exhibit the disadvantage of changing substantially over time under normal storage conditions, in particular as regards their viscosity and their visual appearance.

This is reflected by a cloudy visual appearance and/or an unsatisfactory texture of the compositions, and by a reduced effectiveness.

There thus exists a real need to develop cosmetic compositions, in particular compositions intended for caring for and/or conditioning keratinous fibres, which comprise organosilicon compounds and which do not exhibit the disadvantages described above.

The Applicant Company has now discovered that a composition comprising one or more organosilicon compounds chosen from specific silanes, one or more polysaccharides chosen from anionic and/or non-ionic polysaccharides and one or more water-soluble mineral salts makes it possible to solve the abovementioned disadvantages.

This is because it has been found that the incorporation of a combination of one or more specific polysaccharides and of one or more water-soluble mineral salts in a cosmetic composition comprising one or more organosilicon compounds chosen from specific silanes makes it possible to render these compositions stable on storage, both at ambient temperature (20-25° C.) and at 45° C., in particular as regards their visual appearance and their viscosity, including when these compositions comprise one or more surfactants.

“Stable” within the meaning of the present invention is understood to mean that the visual appearance and the viscosity of these compositions do not change or do not substantially change (variation generally of less than 10%, with respect to the viscosity at T0) over time under standard storage conditions, for example over the month or the two months which follow their manufacture, at ambient temperature. Furthermore, it has also been found that the composition in accordance with the present invention makes it possible to confer good cosmetic properties on the hair.

In particular, it makes it possible to facilitate the shaping of the hair, in particular of fine hair, and to confer improved styling effects, in particular of mass, of body and/or of manageability, in particular on curly hair, in lasting fashion.

The composition according to the invention also has conditioning properties; in particular, it makes it possible to provide the hair with suppleness and smoothness and to facilitate the disentangling thereof. In addition, the composition according to the invention exhibits good use properties, such as a pleasant texture, allowing easy spreading and easy distribution over the hair.

In addition, the said composition can advantageously be transparent. As indicated above, it should be noted that the properties of conditioning and shaping the hair are particularly lasting, and advantageously are persistent towards shampooing operations.

A subject-matter of the present invention is thus a cosmetic composition comprising:
(a) one or more silanes corresponding to the following formula (I) and/or their oligomers:

R₁Si(OR₂)_{z}(R₃)ₓ(OH)_{y} (I)

in which:

R₁ is a linear or branched, saturated or unsaturated, cyclic or acyclic C₁-C₆ hydrocarbon chain substituted by a group chosen from the following groups:
amine NH₂ or NHR, R being a C₁-C₂₀ alkyl, preferably a C₁-C₆ alkyl, optionally substituted by a radical comprising a silicon atom, a C₃-C₄₀ cycloalkyl or a C₆-C₃₀ aromatic,
hydroxyl,
thiol,
aryl or aryloxy which is substituted or unsubstituted, in particular substituted by an amino group or by a C₁-C₄ aminoalkyl group,

it being possible for R₁ to be interrupted by a heteroatom (O, S or NH) or a carbonyl (CO) group,

R₂ and R₃, which are identical or different, represent a linear or branched alkyl group comprising from 1 to 6 carbon atoms,

y denotes an integer ranging from 0 to 3,

z denotes an integer ranging from 0 to 3, and

x denotes an integer ranging from 0 to 2,

with z+x+y=3,
(b) one or more polysaccharides chosen from anionic polysaccharides, non-ionic polysaccharides and their mixtures,
(c) one or more water-soluble mineral salts chosen from halides, sulfates and carbonates.

Another subject-matter of the present invention is a method for the cosmetic treatment of the hair, comprising the application to the said fibres of the composition according to the invention.

Finally, the invention also relates to the use of the composition according to the invention in the treatment of keratinous fibres and in particular in the conditioning and/or care of keratinous fibres, preferably the hair.

Other subject-matters and characteristics, aspects and advantages of the invention will become even more clearly apparent on reading the description and the examples which follow.

As explained above, the composition according to the invention comprises (a) one or more silanes corresponding to the following formula (I) and/or their oligomers:

R₁Si(OR₂)_{z}(R₃)ₓ(OH)_{y} (I)

in which:

R₁ is a linear or branched, saturated or unsaturated, cyclic or acyclic C₁-C₆ hydrocarbon chain substituted by a group chosen from the following groups:
amine NH₂ or NHR, R being a C₁-C₂₀ alkyl, preferably a C₁-C₆ alkyl, optionally substituted by a radical comprising a silicon atom, a C₃-C₄₀ cycloalkyl or a C₆-C₃₀ aromatic,
hydroxyl,
thiol,
aryl or aryloxy which is substituted or unsubstituted, in particular substituted by an amino group or by a C₁-C₄ aminoalkyl group,

it being possible for R₁ to be interrupted by a heteroatom (O, S or NH) or a carbonyl (CO) group,

R₂ and R₃, which are identical or different, represent a linear or branched alkyl group comprising from 1 to 6 carbon atoms,

y denotes an integer ranging from 0 to 3,

z denotes an integer ranging from 0 to 3, and

x denotes an integer ranging from 0 to 2,

with z+x+y=3.

Oligomer is understood to mean the polymerization products of the compounds of formula (I) comprising from 2 to 10 silicon atoms.

Preferably, R₂ represents an alkyl group comprising from 1 to 4 carbon atoms, better still a linear alkyl group comprising from 1 to 4 carbon atoms, and is preferably the ethyl group.

Preferably, R₃ represents an alkyl group comprising from 1 to 4 carbon atoms, better still a linear alkyl group comprising from 1 to 4 carbon atoms, and preferably represents a methyl group or an ethyl group.

Preferably, R₁ is an acyclic chain.

Preferably, the compound of formula (I) comprises only one silicon atom in its structure.

Preferably, z varies from 1 to 3. More preferably still, z is equal to 3 and thus x=y=0.

Preferably, R₁ is a saturated or unsaturated and linear or branched C₁-C₆ hydrocarbon chain substituted by an amine NH₂ or NHR group, R being a C₁-C₂₀ alkyl, preferably a C₁-C₆ alkyl, a C₃-C₄₀ cycloalkyl or a C₆-C₃₀ aromatic.

Preferably, the silane or silanes according to the invention are chosen from 3-aminopropyltriethoxysilane (APTES), 2-aminoethyltriethoxysilane (AETES), 3-aminopropylmethyldiethoxysilane, N-(2-aminoethyl)-3-aminopropyltriethoxysilane, 3-(m-aminophenoxy)propyltrimethoxysilane, p-aminophenyltrimethoxysilane, N-(2-aminoethylaminomethyl)phenethyltrimethoxysilane, their oligomers and a mixture of these compounds, preferably from 3-aminopropyltriethoxysilane (APTES), 2-amino ethyltriethoxysilane (AETES), 3-aminopropylmethyldiethoxysilane, N-(2-aminoethyl)-3-aminopropyltriethoxysilane, their oligomers and a mixture of these compounds, and in particular the silane is chosen from 3-aminopropyltriethoxysilane (APTES), its oligomers and a mixture of these compounds.

According to a preferred embodiment, the silane or silanes are chosen from the compounds of following formula (II):

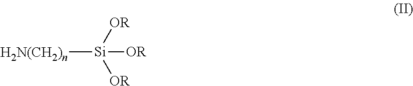

in which the R groups, which are identical or different, are chosen from linear or branched C₁-C₆ alkyl groups and n is an integer ranging from 1 to 6 and preferably from 2 to 4.

A silane which is particularly preferred according to this embodiment is 3-aminopropyltriethoxysilane (APTES). Such a compound is, for example, sold under the name Z-6011 Silane by Dow Corning.

The said silane or silanes used in the composition according to the invention can represent from 0.1% to 20% by weight, preferably from 0.2% to 10% by weight and in particular from 0.4% to 5% by weight, with respect to the total weight of the composition.

As explained above, the composition according to the invention also comprises (b) one or more polysaccharides chosen from anionic polysaccharides, non-ionic polysaccharides and their mixtures.

Mention may in particular be made, as polysaccharides according to the invention, of anionic or non-ionic microbial gums.

Within the meaning of the present invention, “microbial gums” is understood to mean substances synthesized by fermentation of sugars by microorganisms.

The microbial gums can be chosen from scleroglucan gums, gellan gums, pullulan gums, curdlan gums, xanthan gums, grifolan gums, lentinan gums, schizophyllan gums, spirulinan gums and krestin gums.

Mention may in particular be made of the scleroglucan gums produced by Sclerotium rolfsii, the gellan gums produced by Pseudomonas elodea or Sphingomonias, the pullulan gums produced by Aureobacidium pullulens, the curdlan gums produced by Alcaligenes of Faecalis myxogenes type, the xanthan gums produced by numerous organisms, including Leuconostoc mesenteroides and Leuconostoc dextrantum, the grifolan gums produced by Grifola frondara, the lentinan gums produced by Lentinus edodes, the schizophyllan gums produced by Schizophyllum commine, the spirulinan gums produced by Spirulina sybsyla and the krestin gums produced by Coriates versicolor.

Mention may also, in particular, be made of the xanthan gums produced by the bacterium Xanthomonas campestri and the mutants and variants of the latter. These xanthan gums generally have a molecular weight of between 1 000 000 and 50 000 000.

Preferably, use is made, as anionic polysaccharides, of xanthan and scleroglucan gums. More preferably still, use is made of xanthan gums.

Mention may also be made, as polysaccharides according to the invention, of those chosen from glucans, modified or unmodified starches (such as those resulting, for example, from cereals, such as wheat, maize or rice, from vegetables, such as yellow pea, or from tubers, such as potato or cassava), amylose, amylopectin, glycogen, dextrans, celluloses and their derivatives (methylcelluloses, hydroxyalkylcelluloses, ethylhydroxyethylcelluloses, carboxymethylcelluloses), mannans, xylans, lignins, arabans, galactans, galacturonans, chitin, chitosans, glucuronoxylans, arabinoxylans, xyloglucans, glucomannans, pectic acids and pectins, arabinogalactans, carrageenans, agars, gums arabic, gums tragacanth, ghatti gums, karaya gums, locust bean gums, galactomannans, such as guar gums and their non-ionic derivatives (hydroxypropyl guar), and their mixtures.

Mention may be made, as anionic starches, of carboxymethyl starch or starch phosphate.

Use will preferably be made, as non-ionic polysaccharides, of starches, guar gums, celluloses and their derivatives.

Preference will be given, among anionic polysaccharides, to xanthan gum and scleroglucans.

The polysaccharides according to the invention may be modified or unmodified.

The unmodified guar gums are, for example, the products sold under the name Vidogum GH 175 by Unipectine and under the names Meypro-Guar 50 and Jaguar C by Rhodia Chimie.

The modified non-ionic guar gums are in particular modified by C₁-C₆ hydroxyalkyl groups.

Mention may be made, among the hydroxyalkyl groups, by way of example, of the hydroxymethyl, hydroxyethyl, hydroxypropyl and hydroxybutyl groups.

These guar gums are well known from the state of the art and can, for example, be prepared by reacting the corresponding alkene oxides, such as, for example, propylene oxides, with the guar gum, so as to obtain a guar gum modified by hydroxypropyl groups.

The degree of hydroxyalkylation, which corresponds to the number of alkylene oxide molecules consumed by the number of free hydroxyl functional groups present on the guar gum, preferably varies from 0.4 to 1.2.

Such non-ionic guar gums optionally modified by hydroxyalkyl groups are, for example, sold under the trade names Jaguar HP8, Jaguar HP60 and Jaguar HP120, Jaguar DC 293 and Jaguar HP 105 by Rhodia Chimie or under the name Galactasol 4H4FD2 by Aqualon.

Use is made in particular, among the celluloses, of hydroxyethylcelluloses and hydroxypropylcelluloses. Mention may be made of the products sold under the names Klucel EF, Klucel H, Klucel LHF, Klucel MF and Klucel G by Aqualon and Cellosize Polymer PCG-10 by Amerchol.

Preferably, the polysaccharide or polysaccharides used according to the invention are chosen from microbial gums, starches, guar gums, celluloses and their derivatives.

Particularly preferably, the polysaccharide used according to the invention is xanthan gum.

The polysaccharide or polysaccharides can represent from 0.01% to 20% by weight, preferably from 0.1% to 10% by weight and in particular from 0.2% to 5% by weight, with respect to the total weight of the composition.

As explained above, the composition according to the invention also comprises (c) one or more water-soluble mineral salts chosen from halides, sulfates, and carbonates.

Mineral according to the present invention is understood to mean the fact that the salt comprises at most only a single carbon atom, which includes the carbonates.

The water-soluble mineral salt or salts are preferably chosen from salts of mono- or divalent metals, in particular alkali metals or alkaline earth metals, and ammonium salts and in particular from sodium, potassium, magnesium and ammonium salts.

The salts can be in particular chlorides.

Preferably, the mineral salt or salts are chosen from alkali metal or alkaline earth metal chlorides. Mention may in particular be made of sodium chloride, ammonium chloride or potassium chloride; more particularly, the mineral salt is sodium chloride.

The water-soluble mineral salt or salts according to the invention can represent from 0.05% to 20% by weight, preferably from 0.1% to 10% by weight and in particular from 0.2% to 5% by weight, with respect to the total weight of the composition.

Preferably, the composition according to the invention can additionally comprise one or more polyols, in particular one or more polyethylene glycols.

The polyethylene glycol or glycols which can be used in the composition according to the invention preferably comprise from 10 to 500 ethylene glycol units and in particular from 40 to 200 ethylene glycol units. When they are present, the said polyol or polyols, in particular the said polyethylene glycol or glycols according to the invention, can represent from 0.01% to 5% by weight, preferably from 0.05% to 2% by weight and in particular from 0.1% to 1% by weight, with respect to the total weight of the composition.

The composition according to the invention can comprise one or more pH adjusters.

The pH adjusters used according to the invention are preferably chosen from alkaline agents, such as, for example, aqueous ammonia, monoethanolamine, diethanolamine, triethanolamine, 1,3-propanediamine or an alkaline hydroxide, such as 2-amino-2-methyl-1-propanol, or else from acidifying agents, such as, for example, organic acids.

Mention may in particular be made, as organic acid, of acetic acid, propanoic acid, butanoic acid, lactic acid, glycolic acid, ascorbic acid, maleic acid, phthalic acid, succinic acid, taurine, tartaric acid, gluconic acid, glucuronic acid and citric acid.

The pH of the composition used according to the invention is generally between 2 and 10 and preferably between 3 and 8.

The composition according to the invention can also comprise one or more surfactants preferably chosen from non-ionic or cationic surfactants, and their mixtures.

The non-ionic surfactant or surfactants which can be used in the cosmetic composition according to the invention are described, for example, in the Handbook of Surfactants by M. R. Porter, published by Blackie & Son (Glasgow and London), 1991, pp. 116-178. They are chosen in particular from alcohols, α-diols or (C₁-C₂₀)alkylphenols, these compounds being polyethoxylated, polypropoxylated and/or polyglycerolated and having at least one fatty chain comprising, for example, from 8 to 18 carbon atoms, it being possible for the number of ethylene oxide and/or propylene oxide groups to range in particular from 1 to 100 and it being possible for the number of glycerol groups to range in particular from 1 to 30.

Mention may also be made of copolymers of ethylene oxide and of propylene oxide, polyoxyalkylenated fatty acid esters, optionally oxyalkylenated alkyl polyglycosides, alkyl glucoside esters, derivatives of N-alkylglucamine and of N-acylmethylglucamine, aldobionamides, oxycthylenated oils and amine oxides.

Unless otherwise mentioned, “fatty” compound (for example, a fatty acid) denotes, for these surfactants, a compound comprising, in its main chain, at least one saturated or unsaturated alkyl chain comprising at least 6 carbon atoms, preferably from 8 to 30 carbon atoms and better still from 10 to 22 carbon atoms.

“Cationic surfactant” is understood to mean a surfactant which is positively charged when it is present in the composition according to the invention. This surfactant can carry one or more permanent positive charges or can comprise one or more functional groups which can be converted to cations within the composition according to the invention.

The cationic surfactant or surfactants are preferably chosen from primary, secondary or tertiary fatty amines, which are optionally polyoxyalkylenated, or their salts, and quaternary ammonium salts, and their mixtures.

The fatty amines generally comprise at least one C₈-C₃₀ hydrocarbon chain. Mention may be made, among the fatty amines which can be used according to the invention, for example, of stearylamidopropyldimethylamine and distearylamine.

Mention may in particular be made, as quaternary ammonium salts, for example, of:
those corresponding to the following general formula (III):

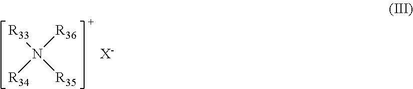

in which the R₃₃ to R₃₆ groups, which can be identical or different, represent a linear or branched aliphatic group comprising from 1 to 30 carbon atoms or an aromatic group, such as aryl or alkylaryl, at least one of the R₃₃ to R₃₆ groups denoting a group comprising from 8 to 30 carbon atoms, preferably from 12 to 24 carbon atoms. The aliphatic groups can comprise heteroatoms, such as, in particular, oxygen, nitrogen, sulfur and halogens. The aliphatic groups are, for example, chosen from C₁-C₃₀ alkyl, C₂-C₃₀ alkenyl, C₁-C₃₀ alkoxy, polyoxy(C₂-C₆)alkylene, C₁-C₃₀ alkylamide, (C₁₂-C₂₂)alkylamido(C₂-C₆)alkyl, (C₁₂-C₂₂)alkyl acetate and C₁-C₃₀ hydroxyalkyl groups; X⁻ is an anion chosen from the group of the halides, phosphates, acetates, lactates, (C₁-C₄)alkyl sulfates, (C₁-C₄)alkylsulfonates or (C₁-C₄)alkylarylsulfonates.

Preference is given, among the quaternary ammonium salts of formula (III), on the one hand, to tetraalkylammonium salts, such as, for example, dialkyldimethylammonium or alkyltrimethylammonium salts in which the alkyl group comprises approximately from 12 to 22 carbon atoms, in particular behenyltrimethylammonium, distearyldimethylammonium, cetyltrimethylammonium or benzyldimethylstearylammonium salts, or, on the other hand, to the palmitylamidopropyltrimethylammonium salt, the stearamidopropyltrimethylammonium salt or the stearamidopropyldimethylcetearylammonium salt. It is particularly preferred to use the salts in the chloride form of these compounds.
quaternary ammonium salts of imidazoline, such as, for example, those of following formula (IV):

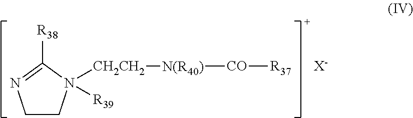

in which R₃₇ represents an alkenyl or alkyl group comprising from 8 to 30 carbon atoms, for example derived from tallow fatty acids, R₃₈ represents a hydrogen atom, a C₁-C₄ alkyl group or an alkenyl or alkyl group comprising from 8 to 30 carbon atoms, R₃₉ represents a C₁-C₄ alkyl group, R₄₀ represents a hydrogen atom or a C₁-C₄ alkyl group and X⁻ is an anion chosen from the group of the halides, phosphates, acetates, lactates, alkyl sulfates, alkylsulfonates or alkylarylsulfonates, the alkyl and aryl groups of which preferably comprise, respectively, from 1 to 20 carbon atoms and from 6 to 30 carbon atoms. Preferably, R₃₇ and R₃₈ denote a mixture of alkenyl or alkyl groups comprising from 12 to 21 carbon atoms, for example derived from tallow fatty acids, R₃₉ denotes a methyl group and R₄₀ denotes a hydrogen atom. Such a product is sold, for example, under the name Rewoquat® W 75 by Rewo;
quaternary diammonium or triammonium salts, in particular of formula (V):

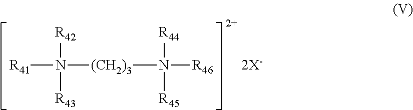

in which R₄₁ denotes an alkyl radical comprising approximately from 16 to 30 carbon atoms, which is optionally hydroxylated and/or interrupted by one or more oxygen atoms, R₄₂ is chosen from hydrogen or an alkyl radical comprising from 1 to 4 carbon atoms or an (R₄₁ₐ)(R₄₂ₐ)(R₄₃ₐ)N—(CH₂)₃ group;

R₄₁ₐ, R₄₂ₐ, R₄₃ₐ, R₄₃, R₄₄, R₄₅ and R₄₆, which are identical or different, are chosen from hydrogen or an alkyl radical comprising from 1 to 4 carbon atoms, and X⁻ is an anion chosen from the group of the halides, acetates, phosphates, nitrates and methyl sulfates. Such compounds are, for example, Finquat CT-P, provided by Finetex (Quaternium 89), or Finquat CT, provided by Finetex (Quaternium 75);
quaternary ammonium salts comprising at least one ester functional group, such as those of following formula (VI):

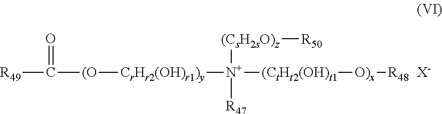

in which:

R₄₇ is chosen from C₁-C₆ alkyl groups and C₁-C₆ hydroxyalkyl or dihydroxyalkyl groups;

R₄₈ is chosen from:
the group

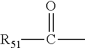

R₅₂ groups, which are saturated or unsaturated and linear or branched C₁-C₂₂ hydrocarbon groups,
the hydrogen atom,

R₅₀ is chosen from:
the group

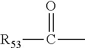

R₅₄ groups, which are saturated or unsaturated and linear or branched C₁-C₆ hydrocarbon groups,
the hydrogen atom,

R₄₉, R₅₁ and R₅₃, which are identical or different, are chosen from saturated or unsaturated and linear or branched C₇-C₂₁ hydrocarbon groups;

r, s and t, which are identical or different, are integers having values from 2 to 6;

r1, r2, t1 and t2, which are identical or different, are integers having values from 0 to 12; with the provisos that r1+r2=2r and t1+t2=2t;

y is an integer having a value from 1 to 10;

x and z, which are identical or different, are integers having values from 0 to 10;

X⁻ is a simple or complex and organic or inorganic anion;

with the proviso that the sum x+y+z has a value from 1 to 15, that, when x has the value 0, then R₄₈ denotes R₅₂ and that, when z has the value 0, then R₅₀ denotes R₅₄.

The R₄₇ alkyl groups can be linear or branched and more particularly linear.

Preferably, R₄₇ denotes a methyl, ethyl, hydroxyethyl or dihydroxypropyl group and more particularly a methyl or ethyl group.

Advantageously, the sum x+y+z has a value from 1 to 10.

When R₄₈ is an R₅₂ hydrocarbon group, it can be long and have from 12 to 22 carbon atoms or be short and have from 1 to 3 carbon atoms.

When R₅₀ is an R₅₄ hydrocarbon group, it preferably has from 1 to 3 carbon atoms.

Advantageously, R₄₉, R₅₁ and R₅₃, which are identical or different, are chosen from saturated or unsaturated and linear or branched C₁₁-C₂₁ hydrocarbon groups and more particularly from saturated or unsaturated and linear or branched C₁₁-C₂₁ alkyl and alkenyl groups.

Preferably, x and z, which are identical or different, have the values 0 or 1.

Advantageously, y is equal to 1.

Preferably, r, s and t, which are identical or different, have the values 2 or 3 and more particularly still are equal to 2.

The anion X⁻ is preferably a halide (chloride, bromide or iodide) or an alkyl sulfate, more particularly methyl sulfate. However, it is possible to use methanesulfonate, phosphate, nitrate, tosylate, an anion derived from an organic acid, such as acetate or lactate, or any other anion compatible with the ammonium comprising an ester functional group.

The anion X⁻ is even more particularly chloride or methyl sulfate.

Use is made more particularly, in the composition according to the invention, of the ammonium salts of formula (VI) in which:

R₄₇ denotes a methyl or ethyl group;

x and y are equal to 1;

z is equal to 0 or 1;

r, s and t are equal to 2;

R₄₈ is chosen from:
the group

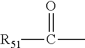

methyl, ethyl or C₁₄-C₂₂ hydrocarbon groups,
the hydrogen atom;

R₅₀ is chosen from:
the group

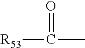

the hydrogen atom;

R₄₉, R₅₁ and R₅₃, which are identical or different, are chosen from saturated or unsaturated and linear or branched C₁₃-C₁₇ hydrocarbon groups and preferably from saturated or unsaturated and linear or branched C₁₃-C₁₇ alkyl and alkenyl groups.

Advantageously, the hydrocarbon groups are linear.

Mention may be made, for example, of the compounds of formula (VI), such as diacyloxyethyldimethylammonium, diacyloxyethyl(hydroxyethyl)methylammonium, monoacyloxyethyldi(hydroxyethyl)methylammonium, triacyloxyethylmethylammonium and monoacyloxyethyl(hydroxyethyl)dimethylammonium salts (in particular chloride or methyl sulfate) and their mixtures. The acyl groups preferably have from 14 to 18 carbon atoms and more particularly originate from a vegetable oil, such as palm oil or sunflower oil. When the compound comprises several acyl groups, the latter can be identical or different.

These products are obtained, for example, by direct esterification of triethanolamine, triisopropanolamine, alkyldiethanolamine or alkyldiisopropanolamine, which are optionally oxyalkylenated, with C₁₀-C₃₀ fatty acids or with mixtures of C₁₀-C₃₀ fatty acids of vegetable or animal origin, or by transesterification of their methyl esters. This esterification is followed by a quaternization using an alkylating agent, such as an alkyl halide (preferably methyl halide or ethyl halide), a dialkyl sulfate (preferably dimethyl sulfate or diethyl sulfate), methyl methanesulfonate, methyl para-toluenesulfonate, glycol chlorohydrin or glycerol chlorohydrin.

Such compounds are, for example, sold under the names Dehyquart® by Henkel, Stepanquat® by Stepan, Noxamium® by Ceca or Rewoquat® WE 18 by Rewo-Witco.

The composition according to the invention can comprise, for example, a mixture of quaternary ammonium mono-, di- and triester salts with a predominance by weight of diester salts.

Use may also be made of the ammonium salts comprising at least one ester functional group described in U.S. Pat. No. 4,874,554 and U.S. Pat. No. 4,137,180.

Use may be made of behenoylhydroxypropyltrimethylammonium chloride, provided by Kao under the name Quatarmin BTC 131.

Preferably, the ammonium salts comprising at least one ester functional group comprise two ester functional groups.

Preference is given, among the quaternary ammonium salts comprising at least one ester functional group which can be used according to the invention, to the use of dipalmitoylethylhydroxyethylmethylammonium salts.

The composition can also comprise anionic surfactants and/or amphoteric surfactants; however, the surfactants are preferably non-ionic or cationic.

When they are present, the surfactants can be present in an amount varying from 0.05% to 30% by weight, preferably from 0.01% to 15% by weight, better still from 0.1% to 10% by weight and even better still from 1% to 8% by weight, with respect to the total weight of the composition.

The composition according to the invention generally comprises at least one solvent chosen from water, C₁-C₈ alcoholic solvents and their mixtures.

The C₁-C₈ alcoholic solvents are generally chosen from alkanols, alkanediols, benzyl alcohol or phenylethyl alcohol.

Preferably, the solvent or solvents are chosen from water and ethanol.

Preferably, the composition according to the invention comprises water in an amount of 50% to 99% by weight, preferably of 55% to 98% by weight, with respect to the total weight of the composition.

The cosmetic composition according to the invention can, in addition, also comprise at least one additive chosen from agents for combating hair loss, antidandruff agents, natural and synthetic thickeners other than those mentioned above, suspending agents, sequestering agents, reducing agents, opacifying agents, colorants, sunscreens, vitamins or provitamins, fragrances and preservatives, and their mixtures.

A person skilled in the art will take care to choose the optional additives and their amounts so that they do not harm the properties of the compositions of the present invention.

The composition according to the invention can be provided in all the forms possible for application to the hair, in particular in the form of a solution of the lotion or serum type, in the form of a gel, in the form of a water-in-oil, oil-in-water or multiple emulsion with a more or less thick liquid consistency, such as more or less smooth milks and creams, or in the form of foams.

The composition according to the invention can be a lotion, a gel, a styling foam or cream, a care cream, a shampoo, a conditioner or a dyeing composition.

Preferably, the composition according to the invention is a care cream and/or a conditioner to be rinsed out or left in.

Preferably, the composition according to the invention is transparent or translucent, in particular when it does not comprise opacifying compounds. Advantageously, it is provided in the form of an aqueous or aqueous/alcoholic gel, in particular one which is translucent or transparent.

Another subject-matter of the invention is a method for the cosmetic treatment of keratinous fibres, in particular human keratinous fibres, such as the hair, in which the cosmetic composition as defined above is applied to the said fibres.

This application may or may not be followed by a rinsing operation. Preferably, it is not followed by a rinsing stage.

When the application of the composition is followed by a rinsing operation, the leave-in time of the composition on the keratinous substances ranges from a few seconds to 60 minutes, better still from 5 seconds to 30 minutes and even better still from 10 seconds to 10 minutes.

The application of the composition according to the invention to hair can be carried out on dry hair or on wet hair. It can in particular be carried out after a shampooing operation or after a pretreatment at acidic or basic pH.

Another subject-matter of the invention relates to the use of the composition as described above in the treatment of keratinous substances, in particular in the conditioning and/or care of keratinous fibres, in particular the hair and especially damaged, very curly, dry or fine hair.

The following examples serve to illustrate the invention without, however, exhibiting a limiting nature.

### EXAMPLES

### Example 1

The composition (A) according to the invention and the comparative composition (A′) are prepared from the ingredients shown in the table below, the amounts of which are expressed as grams of active material.

Amount
(in g)

| Ingredients | A | A′ |
|---|---|---|
| 3-Aminopropyltriethoxysilane (Silsoft A- | 0.5 | 0.5 |
| 1100 from Momentive) | | |
| Xanthan gum (Rhodicarc CFT from | 0.45 | 0.45 |
| Rhodia) | | |
| Sodium chloride | 0.5 | — |
| Polyethylene glycol (180 EO) | 0.2 | 0.2 |
| Lactic acid | 0.32 | 0.32 |
| Glycerol | 5 | 5 |
| 2-Phenoxyethanol | 0.7 | 0.7 |
| Octane-1,2-diol | 0.2 | 0.2 |
| Oxyethylenated (40 EO) hydrogenated | 1.2 | 1.2 |
| castor oil | | |
| Coloured spheres (Unisphercs NT-2302 | 0.15 | 0.15 |
| LO from Induchem) | | |
| Fragrance | 0.3 | 0.3 |
| Deionized water | 90.48 | 90.98 |

The compositions A and A′ can be obtained by dissolution of the silane in the deionized water. This preparation is subsequently left at ambient temperature for a period of time of 30 minutes, and then the polysaccharide and the lactic acid are added to the preparation with continuous stirring in order to homogenize the composition. In the case of the composition A, the mineral salt is then added. The other compounds of the compositions are subsequently added and the compositions A and A′ are thus obtained.

The pH of the two compositions is approximately 4.9.

The composition A is clear (transparent) and fluid, whereas the composition A′ is fluid but translucent (off-white). Both have non-tacky textures to the touch.

After storing for one month at 4° C., the comparative composition A′ forms a gel which is no longer translucent, with a cloudy appearance, and which in addition is very tacky to the touch. For its part, the composition A according to the invention retains the same properties as at the start, whether regarding the transparency or the viscosity; it remains fluid.

The two compositions are compared on a half-head of shoulder-length Caucasian hair. The head of hair is shampooed with a conventional shampoo. The compositions are spread over half-heads in a proportion of 2 g of composition per half-head. The heads of hair are then dried and are subjected to blow drying in the normal way.

The half-heads treated with the composition A according to the invention exhibit an improved suppleness and an improved smoothness to the touch, in comparison with the comparative composition A′, which catches more when the fingers are passed through the head of hair, more particularly on dry hair. The disentangling is also improved with the composition according to the invention.

### Example 2

The composition B according to the invention is prepared from the ingredients shown in the table below, the amounts of which are expressed as grams of active material.

| Ingredients | Amount (in g) |
|---|---|
| 3-Aminopropyltriethoxysilane (Silsoft A- | 0.5 |
| 1100 from Momentive) | |
| Xanthan gum (Rhodicare CFT from | 0.4 |
| Rhodia) | |
| Sodium chloride | 0.5 |
| Polyethylene glycol (180 EO) | 0.2 |
| Lactic acid | 0.32 |
| Glycerol | 5 |
| 2-Phenoxyethanol | 0.7 |
| Octane-1,2-diol | 0.2 |
| Oxyethylenated (40 EO) hydrogenated | 2.6 |
| castor oil | |
| Cetyltrimethylammonium chloride | 0.04 |
| Fragrance | q.s. |
| Deionized water | q.s. for 100 |

The head of hair is shampooed with a conventional shampoo. The composition is spread over a half-head in a proportion of 2 g per half-head, the other half-head being treated with water. The heads of hair are then dried and are subjected to blow drying in the normal way.

The hair treated with the composition according to the invention exhibits, unlike that treated solely with the water, a soft, clean and natural feel, with a non-dry and non-greasy appearance. Furthermore, it is easy to style.

### Example 3

The compositions C to I according to the invention are prepared from the ingredients shown in the table below, the amounts of which are expressed as grams of active material.

| | Amount (in g) | | | |
|---|---|---|---|---|
| Ingredients | C | D | E | F |
| 3-Aminopropyltriethoxysilane (Silsoft A- | 0.5 | 0.3 | 0.4 | 1 |
| 1100 from Momentive) | | | | |
| Xanthan gum (Rhodicare CFT from | 0.05 | — | — | — |
| Rhodia) | | | | |
| Pregelatinized maize hydroxypropyl | — | 0.5 | — | — |
| distarch phosphate | | | | |
| Hydroxyethylcellulose | 0.5 | — | — | — |
| Modified potato starch | — | — | 0.6 | — |
| Starch acetate | — | — | — | 0.6 |
| Sodium chloride | 0.5 | 0.5 | 0.5 | 0.5 |
| Polyethylene glycol (180 EO) | 0.2 | 0.2 | 0.2 | 0.2 |
| Lactic acid | 0.32 | 0.32 | 0.32 | 0.32 |
| Glycerol | 5 | 5 | 5 | 5 |
| 2-Phenoxyethanol | 0.7 | 0.7 | 0.7 | 0.7 |
| Octane-1,2-diol | 0.2 | 0.2 | 0.2 | 0.2 |
| Oxyethylenated (40 EO) hydrogenated | 1.2 | 1.2 | 1.2 | 1.2 |
| castor oil | | | | |
| Fragrance | q.s. | q.s. | q.s. | q.s. |
| Deionized water | q.s. | q.s. | q.s. | q.s. |
| | for | for | for | for |
| | 100 | 100 | 100 | 100 |

| | Amount (in g) | | |
|---|---|---|---|
| Ingredients | G | H | I |
| 3-Aminopropyltriethoxysilane (Silsoft A- | 0.5 | 0.5 | 0.5 |
| 1100 from Momentive) | | | |
| Xanthan gum (Rhodicare CFT from | 0.5 | 0.5 | 0.5 |
| Rhodia) | | | |
| Zinc gluconate | 0.2 | — | — |
| Sodium benzoate | — | 0.2 | — |
| Calcium chloride | — | — | 0.3 |
| Sodium chloride | 0.3 | 0.3 | 0.3 |
| Polyethylene glycol (180 EO) | 0.2 | 0.2 | 0.2 |
| Lactic acid | 0.32 | 0.32 | 0.32 |
| Glycerol | 5 | 5 | 5 |
| 2-Phenoxyethanol | 0.7 | 0.7 | 0.7 |
| Octane-1,2-diol | 0.2 | 0.2 | 0.2 |
| Oxyethylenated (40 EO) hydrogenated | 1.2 | 1.2 | 1.2 |
| castor oil | | | |
| Fragrance | q.s. | q.s. | q.s. |
| Deionized water | q.s. | q.s. | q.s. |
| | for | for | for |
| | 100 | 100 | 100 |

The compositions thus obtained remain stable on storage: their texture and their clearness do not change over time (1 month).

1-15. (canceled)

16. A cosmetic composition comprising:
(a) at least one silane corresponding to the following formula (I) and/or their oligomers:
R₁Si(OR₂)_{z}(R₃)ₓ(OH)_{y} (I)
in which:
R₁ is a linear or branched, saturated or unsaturated, cyclic or acyclic C₁-C₆ hydrocarbon chain substituted by a group chosen from the following groups:
amine NH₂ or NHR, R being a C₁-C₂₀ alkyl optionally substituted by a radical comprising a silicon atom, a C₃-C₄₀ cycloalkyl or a C₆-C₃₀ aromatic,
hydroxyl,
thiol,
aryl or aryloxy which is substituted or unsubstituted,
it being possible for R₁ to be interrupted by a heteroatom or a carbonyl group,
R₂ and R₃, which are identical or different, represent a linear or branched alkyl group comprising from 1 to 6 carbon atoms,
y denotes an integer ranging from 0 to 3,
z denotes an integer ranging from 0 to 3, and
x denotes an integer ranging from 0 to 2,
with z+x+y=3,
(b) at least one polysaccharide chosen from anionic polysaccharides, non-ionic polysaccharides, or mixtures thereof,
(c) at least one or more water-soluble mineral salts chosen from halides, sulfates or carbonates.

17. The composition according to claim 16, wherein the at least one silane is chosen from 3-aminopropyltriethoxysilane, 2-aminoethyltriethoxysilane, 3-aminopropylmethyldiethoxysilane, N-(2-aminoethyl)-3-aminopropyltriethoxysilane, 3-(m-aminophenoxy)propyltrimethoxysilane, p-aminophenyltrimethoxysilane, N-(2-aminoethylaminomethyl)phenethyltrimethoxysilane, their oligomers or mixtures thereof, from 3-aminopropyltriethoxysilane, 2-aminoethyltriethoxysilane, 3-aminopropylmethyldiethoxysilane, N-(2-aminoethyl)-3-aminopropyltriethoxysilane, their oligomers or mixtures thereof.

18. The composition according to claim 16, wherein the at least one silane is chosen from the compounds of following formula (II): 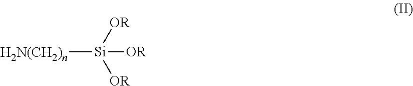
in which the R groups, which are identical or different, are chosen from linear or branched C₁-C₆ alkyl groups and n is an integer ranging from 1 to 6.

19. The composition according to claim 16, wherein the silane is present in an amount ranging from about 0.1% to about 20% by weight, with respect to the total weight of the composition.

20. The composition according to claim 16, wherein the at least one polysaccharide is chosen from microbial gums, scleroglucan gums, gellan gums, pullulan gums, curdlan gums, xanthan gums, grifolan gums, lentinan gums, schizophyllan gums, spirulinan gums, or krestin gums.

21. The composition according to claim 16, wherein the at least one polysaccharide is chosen from glucans, modified or unmodified starches, amylose, amylopectin, glycogen, dextrans, celluloses and their derivatives, mannans, xylans, lignins, arabans, galactans, galacturonans, chitin, chitosans, glucuronoxylans, arabinoxylans, xyloglucans, glucomannans, pectic acids and pectins, arabinogalactans, carrageenans, agars, gums arabic, gums tragacanth, ghatti gums, karaya gums, locust bean gums, galactomannans, guar gums and their non-ionic derivatives, or mixtures.

22. The composition according to claim 16, wherein the at least one polysaccharide is chosen from microbial gums, starches, guar gums, celluloses, or their derivatives.

23. The composition according to claim 16, wherein the polysaccharide is present in an amount ranging from about 0.01% to about 20% by weight, with respect to the total weight of the composition.

24. The composition according to claim 16, wherein the at least one water-soluble mineral salt is chosen from salts of mono- or divalent metals, ammonium salts, sodium salts, potassium salts, or magnesium salts.

25. The composition according to claim 16, wherein the mineral salt is present in an amount ranging from about 0.05% to about 20% by weight, with respect to the total weight of the composition.

26. The composition according to claim 16, further comprising at least one polyol.

27. The composition according to claim 26, wherein the polyol is present in an amount ranging from about 0.01% to about 5% by weight, with respect to the total weight of the composition.

28. The composition according to claim 16, wherein the pH of the composition ranges from about 2 to about 10.

29. A method for the cosmetic treatment of keratinous fibers, in particular human keratinous fibers, the method comprising:
applying to the human keratinous fibers a cosmetic composition comprising:
(a) at least one silane corresponding to the following formula (I) and/or their oligomers:
R₁Si(OR₂)_{z}(R₃)ₓ(OH)_{y} (I)
in which:
R₁ is a linear or branched, saturated or unsaturated, cyclic or acyclic C₁-C₆ hydrocarbon chain substituted by a group chosen from the following groups:
amine NH₂ or NHR, R being a C₁-C₂₀ alkyl optionally substituted by a radical comprising a silicon atom, a C₃-C₄₀ cycloalkyl or a C₆-C₃₀ aromatic,
hydroxyl,
thiol,
aryl or aryloxy which is substituted or unsubstituted,
it being possible for R₁ to be interrupted by a heteroatom or a carbonyl group,
R₂ and R₃, which are identical or different, represent a linear or branched alkyl group comprising from 1 to 6 carbon atoms,
y denotes an integer ranging from 0 to 3,
z denotes an integer ranging from 0 to 3, and
x denotes an integer ranging from 0 to 2,
with z+x+y=3,
(b) at least one polysaccharide chosen from anionic polysaccharides, non-ionic polysaccharides, or mixtures thereof,
(c) at least one or more water-soluble mineral salts chosen from halides, sulfates or carbonates.
